(19) United States
(12) Patent Application Publication
(10) Pub. No.: US 2018/0018757 A1
(43) Pub. Date: Jan. 18, 2018

(54) TRANSFORMING PROJECTION DATA IN TOMOGRAPHY BY MEANS OF MACHINE LEARNING

(71) Applicant: Kenji SUZUKI, HOMEWOOD, IL (US)

(72) Inventor: Kenji SUZUKI, HOMEWOOD, IL (US)

(21) Appl. No.: 15/646,119

(22) Filed: Jul. 11, 2017

Related U.S. Application Data
(60) Provisional application No. 62/362,028, filed on Jul. 13, 2016.

(51) Int. Cl. G06T 3/40; A61B 6/03; G06F 15/18

(52) U.S. Cl. CPC G06T 3/4053; G06F 15/18; A61B 6/03; G06T 7/11

(57) ABSTRACT

A method and system for transforming low-quality projection data into higher quality projection data, using of a machine learning model. Regions are extracted from an input projection image acquired, for example, at a reduced x-ray radiation dose (lower-dose), and pixel values in the region are entered into the machine learning model as input. The output of the machine learning model is a region that corresponds to the input region. The output information is arranged to form an output high-quality projection image. A reconstruction algorithm reconstructs high-quality tomographic images from the output high-quality projection images. The machine learning model is trained with matched pairs of projection images, namely, input lower-quality (lower-dose) projection images together with corresponding desired higher-quality (higher-dose) projection images. Through the training, the machine learning model learns to transform lower-quality (lower-dose) projection images to higher-quality (higher-dose) projection images. Once trained, the trained machine learning model does not require the higher-quality (higher-dose) projection images anymore. When a new lower-quality (low radiation dose) projection image is entered, the trained machine learning model would output a region similar to its desired region, in other words, it would output simulated high-quality (high-dose) projection images where noise and artifacts due to low radiation dose are substantially reduced, i.e., a higher image quality. The reconstruction algorithm reconstructs simulated high-quality (high-dose) tomographic images from the output high-quality (high-dose) projection images. With the simulated high-quality (high-dose) tomographic images, the detectability of lesions and clinically important findings can be improved.

### CROSS-REFERENCE TO RELATED APPLICATIONS

The invention claims the benefit of U.S. application Ser. No. 62/362,028, filed Jul. 13, 2016, entitled “Transforming projection data in tomography by means of machine learning,” which hereby incorporated by reference in its entirety.

### BACKGROUND OF THE INVENTION

### Field

The invention relates generally to the field of tomography and more particularly to techniques, methods, systems, and computer programs for transforming lower quality projection images into higher quality projection images in computed tomography, including but not limited to lower-dose projection images into simulated higher-dose projection images to reconstruct simulated higher-dose tomography images.

This patent specification also generally relates to techniques for processing digital images, for example, as discussed in one or more of U.S. Pat. Nos. 5,751,787; 6,158,888; 6,819,790; 6,754,380; 7,545,965; 9,332,953; 7,327,866; 6,529,575; 8,605,977; and 7,187,794, and U.S. Patent Application No. 2015/0196265; 2017/0071562; and 2017/0178366, all of which are hereby incorporated by reference.

This patent specification includes use of technologies referenced and discussed in the above-noted U.S. Patents and Applications, as well as those discussed in the documents identified in the following List of References, which are cited throughout the specification by reference number (as providing supporting information) and are hereby incorporated by reference:

### LIST OF REFERENCES CITED IN TEXT

[1] D. J. Brenner and E. J. Hall, “Computed tomography—an increasing source of radiation exposure,” N Engl J Med, vol. 357, pp. 2277-84, Nov. 29 2007.
[2] A. Berrington de Gonzalez, M. Mahesh, K. P. Kim, M. Bhargavan, R. Lewis, F. Mettler, et al., “Projected cancer risks from computed tomographic scans performed in the United States in 2007,” Arch Intern Med, vol. 169, pp. 2071-7, Dec. 14 2009.
[3] K. Li, D. Gomez-Cardona, J. Hsieh, M. G. Lubner, P. J. Pickhardt, and G. H. Chen, “Statistical model based iterative reconstruction in clinical CT systems. Part III. Task-based kV/mAs optimization for radiation dose reduction,” Med Phys, vol. 42, p. 5209, September 2015.
[4] S. Pourjabbar, S. Singh, A. K. Singh, R. P. Johnston, A. S. Shenoy-Bhangle, S. Do, et al., “Preliminary results: prospective clinical study to assess image-based iterative reconstruction for abdominal computed tomography acquired at 2 radiation dose levels,” J Comput Assist Tomogr, vol. 38, pp. 117-22, January-February 2014.
[5] S. Singh, M. K. Kalra, S. Do, J. B. Thibault, H. Pien, 0. J. O'Connor, et al., “Comparison of hybrid and pure iterative reconstruction techniques with conventional filtered back projection: dosc reduction potential in the abdomen,” J Comput Assist Tomogr, vol. 36, pp. 347-53, May-June 2012.
[6] M. K. Kalra, M. Woisetschlager, N. Dahlstrom, S. Singh, M. Lindblom, G. Choy, et al., “Radiation dose reduction with Sinogram Affirmed Iterative Reconstruction technique for abdominal computed tomography,” J Comput Assist Tomogr, vol. 36, pp. 339-46, May-June 2012.
[7] P. Prakash, M. K. Kalra, S. R. Digumarthy, J. Hsieh, H. Pien, S. Singh, et al., “Radiation dose reduction with chest computed tomography using adaptive statistical iterative reconstruction technique: initial experience,” J Comput Assist Tomogr, vol. 34, pp. 40-5, January 2010.
[8] A. Padole, S. Singh, D. Lira, M. A. Blake, S. Pourjabbar, R. D. Khawaja, et al.,

“Assessment of Filtered Back Projection, Adaptive Statistical, and Model-Based Iterative Reconstruction for Reduced Dose Abdominal Computed Tomography,” J Comput Assist Tomogr, vol. 39, pp. 462-7, July-August 2015.
[9] Y. Ichikawa, K. Kitagawa, N. Nagasawa, S. Murashima, and H. Sakuma, “CT of the chest with model-based, fully iterative reconstruction: comparison with adaptive statistical iterative reconstruction,” BMC Med Imaging, vol. 13, p. 27, 2013.
[10] R. D. Khawaja, S. Singh, M. Blake, M. Harisinghani, G. Choy, A. Karosmanoglu, et al.,

“Ultralow-Dose Abdominal Computed Tomography: Comparison of 2 Iterative Reconstruction Techniques in a Prospective Clinical Study,” J Comput Assist Tomogr, vol. 39, pp. 489-98, July-August 2015.
[11] R. D. Khawaja, S. Singh, M. Gilman, A. Sharma, S. Do, S. Pourjabbar, et al., “Computed tomography (CT) of the chest at less than 1 mSv: an ongoing prospective clinical trial of chest CT at submillisievert radiation doses with iterative model image reconstruction and iDose4 technique,” J Comput Assist Tomogr, vol. 38, pp. 613-9, July-August 2014.
[12] S. Pourjabbar, S. Singh, N. Kulkarni, V. Muse, S. R. Digumarthy, R. D. Khawaja, et al., “Dose reduction for chest CT: comparison of two iterative reconstruction techniques,” Acta Radiol, vol. 56, pp. 688-95, June 2015.
[13] A. Neroladaki, D. Botsikas, S. Boudabbous, C. D. Becker, and X. Montet, “Computed tomography of the chest with model-based iterative reconstruction using a radiation exposure similar to chest X-ray examination: preliminary observations,” Eur Radiol, vol. 23, pp. 360-6, February 2013.
[14] C. H. McCollough, L. Yu, J. M. Kofler, S. Leng, Y. Zhang, Z. Li, et al., “Degradation of CT Low-Contrast Spatial Resolution Due to the Use of Iterative Reconstruction and Reduced Dose Levels,” Radiology, vol. 276, pp. 499-506, August 2015.
[15] P. Thomas, A. Hayton, T. Beveridge, P. Marks, and A. Wallace, “Evidence of dose saving in routine CT practice using iterative reconstruction derived from a national diagnostic reference level survey,” Br J Radiol, vol. 88, p. 20150380, September 2015.
[16] F. A. Mettler, Jr., W. Huda, T. T. Yoshizumi, and M. Mahesh, “Effective doses in radiology and diagnostic nuclear medicine: a catalog,” Radiology, vol. 248, pp. 254-63, July 2008.
[17] S. Young, H. J. Kim, M. M. Ko, W. W. Ko, C. Flores, and M. F. McNitt-Gray, “Variability in CT lung-nodule volumetry: Effects of dose reduction and reconstruction methods,” Med Phys, vol. 42, pp. 2679-89, May 2015.
[18] M. O. Wielputz, J. Wroblewski, M. Lederlin, J. Dinkel, M. Eichinger, M. Koenigkam-Santos, et al., “Computer-aided detection of artificial pulmonary nodules using an ex vivo lung phantom: influence of exposure parameters and iterative reconstruction,” Eur J Rudiol, vol. 84, pp. 1005-11, May 2015.
[19] J. M. Kofler, L. Yu, S. Leng, Y. Zhang, Z. Li, R. E. Carter, et al., “Assessment of Low-Contrast Resolution for the American College of Radiology Computed Tomographic Accreditation Program: What Is the Impact of Iterative Reconstruction?,” J Comput Assist Tomogr, vol. 39, pp. 619-23, July-August 2015.
[20] K. Suzuki, S. G. Armato, 3rd, F. Li, S. Sone, and K. Doi, “Massive training artificial neural network (MTANN) for reduction of false positives in computerized detection of lung nodules in low-dose computed tomography,” Med Phys, vol. 30, pp. 1602-17, July 2003.
[21] K. Suzuki, I. Horiba, and N. Sugie, “Efficient approximation of neural filters for removing quantum noise from images,” IEEE Transactions on Signal Processing, vol. 50, pp. 1787-1799, July 2002.
[22] K. Suzuki, I. Horiba, and N. Sugie, “Neural edge enhancer for supervised edge enhancement from noisy images,” IEEE Transactions on Pattern Analysis and Machine Intelligence, vol. 25, pp. 1582-1596, December 2003.
[23] H. Arimura, S. Katsuragawa, K. Suzuki, F. Li, J. Shiraishi, S. Sone, et al., “Computerized scheme for automated detection of lung nodules in low-dose computed tomography images for lung cancer screening,” Academic Radiology, vol. 11, pp. 617-629, June 2004.
[24] F. Li, H. Arimura, K. Suzuki, J. Shiraishi, Q. Li, H. Abe, et al., “Computer-aided detection of peripheral lung cancers missed at CT: ROC analyses without and with localization,” Radiology, vol. 237, pp. 684-90, November 2005.
[25] K. Suzuki, J. Shiraishi, H. Abe, H. MacMahon, and K. Doi, “False-positive reduction in computer-aided diagnostic scheme for detecting nodules in chest radiographs by means of massive training artificial neural network,” Acad Radiol, vol. 12, pp. 191-201, February 2005.
[26] K. Suzuki, H. Abe, F. Li, and K. Doi, “Suppression of the contrast of ribs in chest radiographs by means of massive training artificial neural network,” in Proc. SPIE Medical Imaging (SPIE MI), San Diego, Calif., 2004, pp. 1109-1119.
[27] K. Suzuki, H. Abe, H. MacMahon, and K. Doi, “Image-processing technique for suppressing ribs in chest radiographs by means of massive training artificial neural network (MTANN),” IEEE Trans Med Imaging, vol. 25, pp. 406-16, April 2006.
[28] S. Oda, K. Awai, K. Suzuki, Y. Yanaga, Y. Funama, H. MacMahon, et al., “Performance of radiologists in detection of small pulmonary nodules on chest radiographs: effect of rib suppression with a massive-training artificial neural network,” AJR Am J Roentgenol, vol. 193, pp. W397-402, November 2009.
[29] K. Suzuki, F. Li, S. Sone, and K. Doi, “Computer-aided diagnostic scheme for distinction between benign and malignant nodules in thoracic low-dose CT by use of massive training artificial neural network,” IEEE Transactions on Medical Imaging, vol. 24, pp. 1138-1150, September 2005.
[30] K. Suzuki, D. C. Rockey, and A. H. Dachman, “CT colonography: Advanced computer-aided detection scheme utilizing MTANNs for detection of “missed” polyps in a multicenter clinical trial,” Med Phys, vol. 30, pp. 2-21, 2010.
[31] K. Suzuki, H. Yoshida, J. Nappi, S. G. Armato, 3rd, and A. H. Dachman, “Mixture of expert 3D massive-training ANNs for reduction of multiple types of false positives in CAD for detection of polyps in CT colonography,” Med Phys, vol. 35, pp. 694-703, February 2008.
[32] K. Suzuki, H. Yoshida, J. Nappi, and A. H. Dachman, “Massive-training artificial neural network (MTANN) for reduction of false positives in computer-aided detection of polyps: Suppression of rectal tubes,” Med Phys, vol. 33, pp. 3814-24, October 2006.
[33] J. Xu and K. Suzuki, “Massive-training support vector regression and Gaussian process for false-positive reduction in computer-aided detection of polyps in CT colonography,” Medical Physics, vol. 3R, pp 1888-1902, 2011.
[34] K. Suzuki, J. Zhang, and J. Xu, “Massive-training artificial neural network coupled with Laplacian-eigenfunction-based dimensionality reduction for computer-aided detection of polyps in CT colonography,” IEEE Trans Med Imaging, vol. 29, pp. 1907-17, November 2010.
[35] A. C. Kak, M. Slaney, and IEEE Engineering in Medicine and Biology Society., Principles of computerized tomographic imaging. New York: IEEE Press, 1988.
[36] C. L. Byrne, Applied iterative methods. Wellesley, Mass.: AK Peters, 2008.
[37] V. N. Vapnik, “Problem of Regression Estimation,” in Statistical Learning Theory, ed New York: Wiley, 1998, pp. 26-28.
[38] S. Haykin, “Statistical Nature of Learning Process,” in Neural Networks, ed Upper Saddle River, N.J.: Prentice Hall, 1998, pp. 84-87.
[39] V. N. Vapnik, “SV Machine for Regression Estimation,” in Statistical Learning Theory, ed New York: Wiley, 1998, pp. 549-558.
[40] C. E. Rasmussen, “Gaussian processes for machine learning,” 2006.
[41] V. N. Vapnik, “Least Squares Method for Regression Estimation Problem,” in Statistical Learning Theory, ed New York: Wiley, 1998, p. 34.
[42] S. Haykin, “Back-Propagation Algorithm,” in Neural Networks, ed Upper Saddle River, N.J.: Prentice Hall, 1998, pp. 161-175.

### BACKGROUND

Computed tomography (CT) (also known as computerized axial tomography (CAT)) and various other tomographic imaging techniques such as positron emission tomography (PET) system, a single photon emission computed tomography (SPECT) system, a magnetic resonance imaging (MRI) system, an ultrasound (US) imaging system, an optical coherent tomography system, and tomosynthesis have been used to detect diseases, abnormalities, objects, and defects such as cancer in patients, a defect in an integrated circuit (IC) chip, and a weapon that a person hides.

Because of its necessity, a large number of CT exams are performed. In the U.S., 85 million CT scans are performed each year. CT images, for example, allow patients to screen for tissue anomalies, classifying them based on indicators such as abnormal or normal, lesion or non-lesion, and malignant or benign. In cancer detection in CT, a radiologist assesses volumes of CT image data of a subject tissue. The U.S. Preventive Services Task Force (USPSTF) recommends annual screening for lung cancer with low-dose CT (LDCT) in adults aged 55 to 80 years who have a 30 pack-year smoking history and currently smoke or have quit within the past 15 years.

Given the volume of CT data, however, it can be difficult to identify and fully assess CT image data for cancer detection. CT image analysis is known to result in mis-diagnoses in some instances. A radiologist can miss lesions in CT images, i.e., false negatives, or he/she may detect non-lesions erroneously as lesions. Both such false negatives and false positives lower overall accuracy of detection and diagnosis of lesions with CT images. Image quality of CT images greatly affects the accuracy of detection and diagnosis of lesions. Similarly, in non-medicine areas, image quality of CT images affects the accuracy of a given task that uses CT such as detection of a defect in an integrated circuit (IC) chip and a weapon that a person hides.

There is a tradeoff between radiation dosage levels and image quality, when a radiologist or a computer detects, interprets, analyzes, and diagnoses CT images. The image quality generally affects the accuracy and efficiency in image analysis, interpretation, detection and diagnosis. Higher radiation doses result in higher signal-to-noise ratio with fewer artifacts, while lower doses lead to increased image noise, including quantum noise and electronic noise, and more artifacts. Although high radiation dose produces high image quality, the risk for developing cancer increases. Recent studies [1] show that CT scans in the U.S. might be responsible for up to 1.5-2.0% of cancers [1], and CT scans performed in each year would cause 29,000 new cancer cases in the U.S. due to ionizing radiation exposures to patients [2]. That would result in estimated 15,000 cancer deaths. Increasing use of CT in modern medicine has led to serious public concerns over potential increase in cancer risk from associated radiation doses. Therefore, it is important to reduce radiation exposures and doses as much as possible, or radiation exposures and doses should be kept as low as reasonably achievable. Thus, in clinic, lower radiation dose is used so as not to increase the risk for developing cancer without sacrificing diagnostic quality.

A number of researchers and engineers have developed techniques for radiation dose reduction. There are 3 major categories in the techniques: 1) Acquisition-based techniques such as adaptive exposure; 2) reconstruction-based techniques such as iterative reconstruction; and 3) image-domain-based techniques such as nose-reduction filters. As radiation dose decreases, heavy noise and artifact appear in CT images. In category 3), image-based noise reduction filters are computationally fast, reduce noise but do not reduce artifacts. Recent advances have led to introduction of several technologies to enable CT dose reduction including iterative reconstruction (IR) algorithms [3-12] in category 2), mainstream technology to enable dose reduction by reconstruction of scan raw data. However, reports [13, 14] suggest limitations of IR at very low levels of radiation, such as paint-brush image appearance, blocky appearance of structures, and loss of low contrast resolution structures. A recent national survey study with more than 1,000 hospitals in Australia [15] revealed that the IR technologies reduced radiation dose by 17-44%, which is not sufficient for screening exams. Advanced (or full) IR operate on scan raw data which require substantial expenses in terms of more advanced CT scanners or reconstruction boxes with some limited legacy CT. It took long time to process a case on a standard computer. By developing a specialized massively parallel computer of 112 CPU cores in a large cabinet box, manufactures cut the processing time down to several to dozen minutes per one scan, which is still by far longer than the time when radiologists or patients can wait.

Average radiation dose in a chest CT exam is 7 millisievert (mSv) [16]. IR allow low-dose (LD) CT for lung cancer screening population at approximately 1.0-1.5 mSv levels, but they have limitations of alterations in image texture which often give IR images distracting appearance compared to filtered back projection (FBP) that radiologists have used for the past four decades, although such distracting appearance does not lower the performance of computer analysis [17, 18]. Studies reported that 25% dose reduction with IR resulted in degradation of spatial resolution [14] and contrast [19]. The radiation dose under the current LDCT protocols with IR is still very high for screening population, because annual CT screening will increase cumulative radiation exposure and lifetime attributable risks for radiation-induced cancer.

Thus, despite a number of developments in radiation dose reduction techniques in CT, current radiation dosing is still very high, especially for screening populations. To address this serious issue, the techniques of the present invention provide a way of using low-dose CT imaging with improved, higher-dose like image quality.

On the other hand, a number of researchers have developed automated techniques to analyze CT images. A computer-aided detection (CAD) of lesions in CT aims to automatically detect lesions such as lung nodules in CT images. A computer-aided diagnosis system for lesions in CT is used for assisting radiologists improve their diagnosis. The performance of such computer systems is influenced by the image quality of CT. For example, noise and artifacts in low-dose CT can lower the performance of a computer-aided detection system for lesions in CT.

In the field of CAD, K. Suzuki et al. developed a pixel-based machine-learning technique based on an artificial neural network (ANN), called massive-training ANNs (MTANN), for distinguishing a specific opacity (pattern) from other opacities (patterns) in 2D CT images [20]. An MTANN was developed by extension of neural filters [21] and a neural edge enhancer [22] to accommodate various pattern-recognition and classification tasks [20]. The 2D MTANN was applied to reduction of false positives (FPs) in computerized detection of lung nodules on 2D CT slices in a slice-by-slice way [20, 23, 24] and in chest radiographs [25], the separation of ribs from soft tissue in chest radiographs [26-28], and the distinction between benign and malignant lung nodules on 2D CT slices [29]. For processing of three-dimensional (3D) volume data, a 3D MTANN was developed by extending the structure of the 2D MTANN, and it was applied to 3D CT colonography data [30-34].

Applications of artificial neural network (ANN) techniques to medical pattern recognition and classification, called massive-training ANNs (MTANNs), are discussed in U.S. Pat. Nos. 6,819,790, 6,754,380, 7,545,965, 7,327,866, and 9,332,953, and U.S. Publication No. 2006/0018524. The MTANN techniques of U.S. Patent Nos. 6,819,790, 6,754,380, and U.S. Publication No. 2006/0018524 are developed, designed, and used for pattern recognition or classification, namely, to classify patterns into certain classes, e.g., classification of a region of interest in CT into an abnormal or normal. In other words, the final output of the MTANN is classes such as 0 or 1, whereas the final output of the methods and systems described in this patent specification, the machine-learning model, is continuous values (or images) or pixel values. The techniques of U.S. Pat. No. 7,545,965 are developed, designed, and used for enhancing or suppressing specific patterns such as ribs and clavicles in chest radiographs, whereas the machine-learning models in this invention are used for radiation dose reduction in computed tomography. The techniques of U.S. Pat. No. 9,332,953 are developed, designed, and used for radiation dose reduction specifically for reconstructed computed tomographic images, namely, they do not use or include a reconstruction algorithm in the techniques or do not use or include raw projection images (such as a sinogram) from a detector before reconstruction, but use reconstructed images from a CT scanner (namely, they are outside a CT scanner); whereas the techniques in this present invention are used for image quality improvement in raw projection images before reconstruction, and they use or include a reconstruction algorithm in the method. In other words, the techniques of U.S. Pat. No. 9,332,953 are limited to reconstructed tomographic images in the image domain, namely, an image-domain-based method, whereas the machine-learning models in this invention are used in the raw projection data (such as sinogram) domain, namely, a reconstruction-based method. Also, the techniques of U.S. Pat. No. 9,332,953 are limited to radiation dose reduction and are limited to noise reduction and edge contrast improvement. Because the techniques in this present invention use the original raw projection data that contain all the information acquired with the detector, no information is lost or reduced in the data, whereas image-domain-based methods such as the techniques of U.S. Pat. No. 9,332,953 use reconstructed images which do not contain all the information from the detector (namely, some data are lost or reduced in the process of reconstruction). Therefore, a higher performance can be obtained by using the techniques in this present invention than do image-domain-based methods. The techniques of U.S. Patent Application No. 20150196265 are developed, designed, and used for radiation dose reduction specifically for mammograms, namely, they do not use or include a reconstruction algorithm in the techniques; whereas the machine-learning models in this invention are used for image quality improvement in raw projection images before reconstruction, and they use or include a reconstruction algorithm in the method. The techniques of U.S. Patent Application No. 2017/0071562 are developed, designed, and used for radiation dose reduction specifically for breast tomosynthesis. In other words, the techniques of U.S. patent application Ser. No. 14/596869 and No. 2017/0071562 are limited to breast imaging including mammography and breast tomosynthesis.

The techniques of U.S. Pat. No. 6,529,575 do not use machine learning, but adaptive filtering to reduce noise. The techniques of U.S. Pat. No. 8,605,977 do not use machine learning, but an iterative pixel-wise filter to reduce noise. The techniques of U.S. Pat. No. 7,187,794 do not use machine learning, but a domain specific filter to reduce noise. The techniques of U.S. Patent Application No. 2017/0178366 use voxel-wise iterative operations to reconstruct tomographic images, but not reduce radiation dose.

### BRIEF SUMMARY OF THE INVENTION

This patent application describes transforming lower quality raw projection data (or images or volumes) (for example, sinograms) into higher quality raw projection data (images/volumes) (e.g., sinograms), including but not limited to transforming lower-dose raw projection images with much noise and more artifacts into higher-dose-like raw projection images with less noise or artifacts. The transformed higher-dose-like (or simulated high-dose) raw projection images are subject to a reconstruction algorithm such as back-projection, filtered back-projection (FBP), inverse Radon transform, Fourier-domain reconstruction, iterative reconstruction (IR), maximum likelihood expectation maximization reconstruction, statistical reconstruction techniques, polyenergetic nonlinear iterative reconstruction, likelihood-based iterative expectation-maximization algorithms, algebraic reconstruction technique (ART), multiplicative algebraic reconstruction technique (MART), simultaneous algebraic reconstruction technique (SART), simultaneous multiplicative algebraic reconstruction technique (SMART), pencil-beam reconstruction, fan-beam reconstruction, cone-beam reconstruction, sparse sampling reconstruction, and compress sensing reconstruction. The reconstruction algorithm reconstructs tomographic images from the output simulated high-dose x-ray projection images.

The present technique and system use a machine-learning model with an input local window and an output smaller local window (preferably a single pixel). The input local window extracts regions (image patches or subvolumes) from input raw projection data (images, volumes, or image sequences). The output smaller local windows (preferably, single pixels/voxels) form output raw projection data (image, volume or image sequence). A preferred application example is transforming low dose x-ray projection images (e.g., sinograms) into high-dose-like (simulated high-dose) x-ray projection images (e.g., sinograms). A sinogram is a 2D array of data that contain 1D projections acquired at different angles. In other words, a sinogram is a series of angular projections that is used for obtaining a tomographic image. The output high-dose-like x-ray projection images (e.g., sinograms) are subject to a reconstruction algorithm such as filtered back-projection, inverse radon transform, iterative reconstruction (IR), or algebraic reconstruction technique (ART). The reconstruction algorithm reconstructs tomographic images from the output high-dose-like x-ray projection images (e.g., sinograms). We expect that the reconstructed tomographic images are similar to high-dose computed tomographic images, or simulated high-dose computed tomographic images, where noise or artifacts are substantially reduced.

The machine-learning model in the invention is trained with lower-quality x-ray projection images together with corresponding higher-quality x-ray projection images. In a preferred example, the machine-learning model is trained with lower-radiation-dose projection images (e.g., sinograms) together with corresponding “desired” higher-radiation-dose projection images (e.g., sinograms). After training, the trained machine-learning model would output projection images similar to the “desired” higher-radiation-dose projection images. Then, the reconstruction algorithm reconstructs high-quality tomographic images from the output high-dose-like projection images.

### BRIEF DESCRIPTION OF THE DRAWINGS

FIG. 1 shows a schematic diagram of the imaging chain in a tomographic imaging system, including the present invention of the machine-learning-based transformation.

FIG. 2A shows a schematic diagram of the machine-learning-based transformation in a supervision step.

FIG. 2B shows a schematic diagram of the machine-learning-based transformation in a reconstruction step.

FIG. 3A shows a detailed architecture of the machine-learning model that uses a patch learning machine.

FIG. 3B shows supervision of the machine-learning model in the machine-learning-based transformation.

FIG. 4A shows a flow chart for a supervision step of the machine-learning-based transformation.

FIG. 4B shows a flow chart for a reconstruction step of the machine-learning-based transformation.

FIGS. 5A and 5B show flow charts for a supervision step and a reconstruction step of the machine-learning-based transformation, respectively.

FIG. 6A shows a schematic diagram of the machine-learning-based transformation in a supervision step when a series of 2D raw projection images are acquired from a system.

FIG. 6B shows a schematic diagram of the machine-learning-based transformation in a reconstruction step when a series of 2D raw projection images are acquired from a system.

FIG. 7A shows an example of the training of the machine-learning-based transformation that uses features extracted from local regions (the size of which are not necessarily the same as the input regions) as input.

FIG. 7B shows an example of the machine-learning-based transformation that uses features extracted from local regions (the size of which are not necessarily the same as the input regions) as input.

FIG. 8A shows a schematic diagram of a multiple-machine-learning-based transformation in a supervision step in a multi-resolution approach.

FIG. 8B shows a schematic diagram of a multiple-machine-learning-based transformation in a reconstruction step in a multi-resolution approach.

FIG. 9A shows an ultra-low-dose reconstructed CT image and a simulated high-dose reconstructed CT image obtained by using the trained machine learning model.

FIG. 9B shows corresponding reference-standard real higher-dose reconstructed CT image.

FIG. 10 shows estimates for radiation dose equivalent to that of a real, high-dose CT image by using a relationship between radiation dose and image quality.

FIG. 11 shows an exemplary block diagram of a system that trains the machine learning-based transformation or using a trained machine learning model in the form of a computer.

FIG. 12 shows a schematic diagram of a sequential approach of machine-learning-based transformation in the raw-projection domain followed by machine-learning-based transformation in the reconstructed image domain.

### DETAILED DESCRIPTION OF THE INVENTION

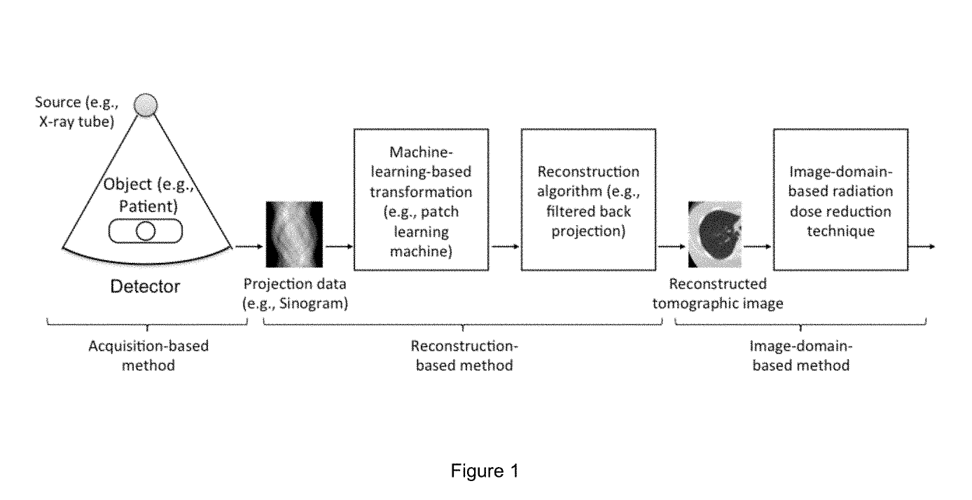

Tomographic imaging systems such as a computed tomography (CT) system acquire raw projection data (signals/images/volumes) where electro-magnetic waves such as x-ray, ordinary light, ultraviolet light, and infrared light, and sound waves such as ultrasound, path through an object to carry the specific information on the object, for example, x-ray carries the information on x-ray attenuation coefficients of the materials in the object. FIG. 1 shows a schematic diagram of the imaging chain in a tomographic imaging system, including the present invention of machine-learning-based transformation. A reconstruction algorithm, such as filtered back-projection, inverse Radon transform, iterative reconstruction (IR), or algebraic reconstruction technique (ART), reconstructs tomographic images from the acquired raw projection signals/images/volumes. There are three classes of image-quality improvement methods in the chain: acquisition-based methods, reconstruction-based methods, and image-domain-based methods. This present invention is in the category of reconstruction-based methods, as opposed to acquisition-based methods or image-domain (or reconstructed-tomographic-image-domain) -based methods. To my knowledge, no machine-learning technique, such as artificial neural networks, support vector machine, support vector regression, shallow or deep convolutional neural networks, deep learning, deep brief networks, and supervised nonlinear regression, has applied to this domain. Unlike acquisition-based methods, this present invention is applied to acquired data, namely, raw projection data from a detector such as a sinogram. Unlike image-domain-based methods, this present invention is applied to raw projection data before reconstructing to form tomographic images.

In preferred examples, this present technique in this invention transforms lower-quality (raw) projection data (signals/images/volumes) (e.g., sinograms) into higher-quality (raw) projection data (signals/images/volumes) (e.g., sinograms), including but not limited to transforming lower-dose raw projection images with much noise and more artifacts into higher-dose-like raw projection images with less noise or artifacts. The transformed higher-dose-like raw projection images are subject to a reconstruction algorithm such as filtered back-projection, inverse Radon transform, iterative reconstruction (IR), or algebraic reconstruction technique (ART). The reconstruction algorithm reconstructs tomographic images from the output high-dose-like x-ray projection images.

The machine-learning model with an input local window and an output local window (preferably a local window smaller than the input local window, at the minimum a single pixel) is used in the present invention. In a preferred example, an artificial neural network regression is used as the machine-learning model. Other machine learning models can be used, including but not limited to support vector regression, supervised nonlinear regression, support vector regression, a nonlinear Gaussian process regression model, shallow or deep convolutional neural network, shift-invariant neural network, deep learning, deep belief networks, nearest neighbor algorithm, association rule learning, inductive logic programming, reinforcement learning, representation learning, similarity learning, sparse dictionary learning, manifold learning, dictionary learning, boosting, Bayesian networks, case-based reasoning, Kernel machines, subspace learning, Naive Bayes classifiers, ensemble learning, random forests, decision trees, a bag of visual words, and statistical relational learning.

The input local window of the machine-learning model extracts regions (or image patches, subvolumes) from input raw projection data (images, volumes, image sequences, or sinograms). The size of the input local window is generally larger than or equal to that of the output local window of the machine-learning model. The input local window shifts in the input raw projection data (images), and the shifted local windows overlap, while the output local window shifts accordingly. The output local window (preferably smaller than the input local window, at the minimum a single pixel/voxel) of the machine-learning model provides regions to form an output raw projection data (image, volume, image sequence, or sinogram).

The output projection images are subject to a tomographic reconstruction algorithm such as back-projection, filtered back-projection (FBP), inverse Radon transform, Fourier-domain reconstruction, iterative reconstruction (IR), maximum likelihood expectation maximization reconstruction, statistical reconstruction techniques, polyenergetic nonlinear iterative reconstruction, likelihood-based iterative expectation-maximization algorithms, algebraic reconstruction technique (ART), multiplicative algebraic reconstruction technique (MART), simultaneous algebraic reconstruction technique (SART), simultaneous multiplicative algebraic reconstruction technique (SMART), pencil-beam reconstruction, fan-beam reconstruction, cone-beam reconstruction, sparse sampling reconstruction, and compress sensing reconstruction. The tomographic reconstruction algorithm reconstructs (N+1) dimensional data from N dimensional projection data. For example, it reconstructs 2-dimensional (2D) structures in a 2D image from a series of 1D projection data measured by rotating the source and detector (or the object). It reconstructs a 3D structures in a 3D volume from measured 2D projection data (or 2D raw projection images). FBP is an analytic, deterministic reconstruction algorithm, but FBP is fully correct only when the noise influence can be neglected and when the number of projections is infinite. Therefore, FBP can lead to artifacts in reconstructed images due to low-radiation-dose-induced noise. The machine learning model prior to the reconstruction algorithm converts low-dose projection images with much noise to high-dose-like projection images with less noise. That allows FBP or other reconstruction algorithms to provide high-quality reconstructed data/images where noise and artifacts are substantially reduced.

There are two main steps associated with the techniques in this present invention: (1) a supervision step to determine the parameters in the machine-learning model to transform lower-quality projection data to higher-quality projection data and (2) a reconstruction step to reconstruct tomographic images from transformed higher-quality projection data. The machine-learning model is trained with lower-quality projection images (e.g., sinograms) together with corresponding “desired” higher-quality projection images (e.g., sinograms). After training, the trained machine-learning model would output projection images similar to the “desired” higher-quality projection images. Then, the reconstruction algorithm reconstructs high-quality tomographic images from the output high-quality projection images.

A preferred application example is transforming low-dose x-ray projection images (e.g., sinograms) into high-dose-like x-ray projection images (e.g., sinograms). Higher radiation doses result in higher signal-to-noise ratio images with less noise or fewer artifacts, whereas lower doses lead to increased noise and more artifacts in projection images, thus lower-dose projection images are of low quality. For this application, the machine-learning model is trained with input lower-dose, lower-quality projection images (e.g., sinograms) with much noise and more artifacts together with the corresponding higher-dose, higher-quality projection images (e.g., sinograms) with less noise or artifacts. Once the machine-learning model is trained, the higher-dose projection images (e.g., sinograms) are not necessary any more, and the trained machine-learning model is applicable to new low-dose projection images to produce the high-dose-like projection images or simulated high-dose projection images where noise and artifacts are substantially reduced. It is expected that high-dose-like projection images look like real high-dose projection images. Then, the output high-dose-like x-ray projection images (e.g., sinograms) are subject to a reconstruction algorithm such as filtered back-projection, inverse radon transform, iterative reconstruction (IR), or algebraic reconstruction technique (ART). The reconstruction algorithm reconstructs tomographic images from the output high-dose-like x-ray projection images (e.g., sinograms). The reconstructed tomographic images are similar to high-dose CT images, or simulated high-dose computed tomographic images where noise or artifacts are removed, or at least substantially reduced. With high image-quality reconstructed images provided by the machine-learning model, radiologists' diagnostic performance, namely, regarding sensitivity and specificity of lesions would be improved; and thus, mortality and incidence of cancer as well as other diseases would potentially be improved with improved tomographic images.

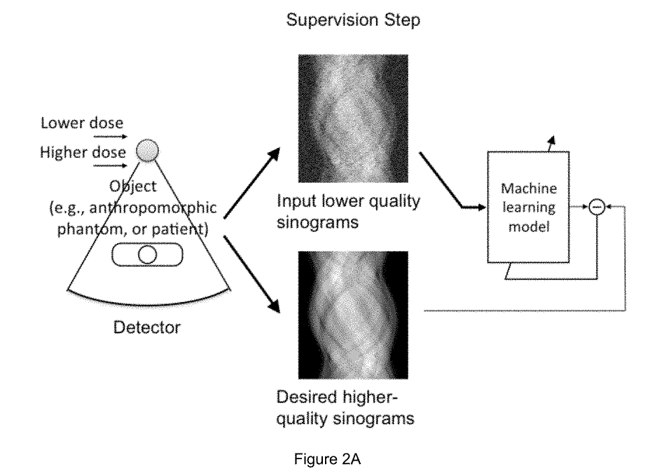

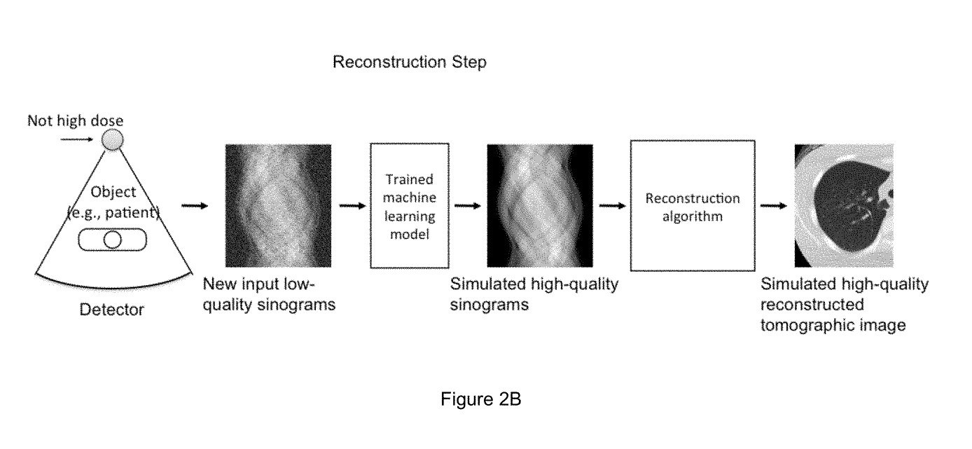

FIG. 2A shows a schematic diagram of the machine-learning-based transformation in a supervision step. In the supervision step, the machine-learning model is supervised with input lower-quality (e.g., lower-dose) projection images with high image degradation factors (e.g., much noise, more artifacts, and/or much blurriness, and/or low-contrast and high-sharpness) and the corresponding desired higher-dose projection images with improved image degradation factors (e.g., less noise, less artifact, and/or less blurriness and/or high-contrast and high-sharpness). The parameters in the machine-learning model are adjusted to minimize the difference between the output projection images and the corresponding desired projection images. Through the supervision process, the machine-learning model learns to convert lower-quality (e.g., lower-dose) projection images with much noise, many artifacts, and much blurriness, and low-contrast to higher-quality-like (e.g., higher-dose-like) projection images with improved image-degradation factors (e.g. less noise, less artifacts, or less blurriness and/or high-contrast and high-sharpness).

The number of supervising input and desired projection images may be relatively small, e.g., 1, 10, or 100 or less. However, a larger number of supervising images may be used as well, e.g., 100-1,000 projection images, 1,000-10,000 projection images, 10,000-100,000 projection images, 100,000-1,000,000 projection images, or more than 10,000,000 projection images.

FIG. 2B shows a schematic diagram of a machine-learning-based transformation in a reconstruction step. Once the machine-learning model is trained, the trained machine-learning model does not require higher-quality (e.g., higher-dose) projection images anymore. When a new lower-quality (e.g., reduced radiation dose or low dose) projection image is entered, the trained machine-learning model would output a projection image similar to its desired projection image, in other words, it would output high-quality (e.g., high-dose-like) projection images or simulated high-dose projection images where image degradation factors such as noise, artifact, and blurriness due to low radiation dose are substantially reduced (or improved).

In the application to radiation dose reduction, projection images acquired at a low radiation dose level have much noise. The noise in low-dose projection images contains two different types of noise: quantum noise and electronic noise. Quantum noise is modeled as signal-dependent noise, and electronic noise is modeled as signal-independent noise. The machine-learning model is expected to eliminate or at least substantially reduce both quantum noise and electronic noise. In addition to noise characteristics, the conspicuity of objects (such as lesions, anatomic structures, and soft tissue) in higher-dose projection images is higher than that of such objects in lower-dose projection images. Therefore, the machine-learning model is expected to improve the conspicuity of such objects (e.g., normal and abnormal structures) in projection images.

The simulated high-dose projection images are then subject to a tomographic reconstruction algorithm such as filtered back-projection (FBP), inverse Radon transform, iterative reconstruction (IR), or algebraic reconstruction technique (ART). The tomographic reconstruction algorithm reconstructs (N+1) dimensional data from N dimensional projection data. For example, it reconstructs 3D structures in a simulated high-dose 3D volume with less noise or artifacts from a series of the simulated high-dose 2D projection images, or it reconstructs a simulated high-dose 2D image with less noise or artifacts from a simulated high-dose sinogram.

To briefly describe tomographic reconstruction and the theory behind it, let us define the projection of objects, resulting from the tomographic measurement process at a given angle θ, is made up of a set of line integrals. The line integral represents the total attenuation of the beam of x-rays as it travels in a straight line through the object. The total attenuation p of an x-ray at position r, on the projection at angle θ, is given by the line integral

$${p}_{θ} ( r ) = {\int }_{- ∞}^{∞} {\int }_{- ∞}^{∞} f ( x , y ) δ ( x cos θ + y sin θ - r ) dxdy , \qquad ( 1 )$$

where δ is the Dirac delta function, and f(x,y) is a 2D tomographic image that we wish to find. This equation is known as the Radon transform of the function f(x,y). The inverse transformation of the Radon transform is called the inverse Radon transform or the back projection, represented by

$$\begin{matrix}f ( x , y ) = {\int }_{0}^{π} {Q}_{θ} ( x cos θ + y sin θ ) d θ , & ( 2 ) \\ where & \\ {Q}_{θ} ( r ) = {\int }_{- ∞}^{∞} {S}_{θ} ( ω ) \vert ω \vert {e}^{j 2 πω r} d ω , & ( 3 )\end{matrix}$$

S_{θ}(ω) is the Fourier transform of the projection at angle θ p_{θ}(r). This way, a 2D tomographic image is reconstructed from a series of 1D projections. Likewise, N+1 dimensional data can be reconstructed from a series of N dimensional projection data. In practical, the filtered back projection method is used to reconstruct images from projection data (the formulation of which is descried in [35], and see pages 49-107 in [35] for details).

An alternative family of tomographic reconstruction algorithms is the algebraic reconstruction techniques (ART). ART can be considered as an iterative solver of a system of linear equations. The values of the pixels are considered as variables collected in a vector x, and the image processing is described by a matrix A. The measured angular projections are collected in a vector b. Given a real or complex m×n matrix A and a real or complex vector b, respectively, the method computes an approximation of the solution of the linear systems of equations. ART can be used for reconstruction of limited projection data (for example, under the situation where project data at all 180 degrees are not acquired, such as a tomosynthesis system). Another advantage of ART over FBP is that it is relatively easy to incorporate prior knowledge into the reconstruction process. The formulation of ART is descried in [35], and see pages 275-296 in [35] for details.

Another approach uses an iterative scheme of tomographic reconstruction, called iterative reconstruction (IR). An IR algorithm is typically based on expectation maximization (EM). In the first iteration, the uniform “trial” object is taken into account and its projections are computed using a physical model. The projections obtained are compared with those acquired by measurement. Using this comparison, the trial object is modified to produce projections that are closer to the measured data. Then, the algorithm iteratively repeats. The trial object is modified in each iteration, and its projections converge to measured data. IR requires heavy computation. To improve the efficiency of IR, a technique of ordered subsets (OS) can be used. When combined with the EM method, it is called OSEM. OS technique splits each iteration into several sub-iterations. In each sub-iteration, just a selected subset of all projections is used for trial object modification. The following sub-iteration uses a different subset of projections and so on. After all projections are used, the single full iteration is finished. The formulation of IR is descried in [36], and see pages 267-274 in [36] for details.

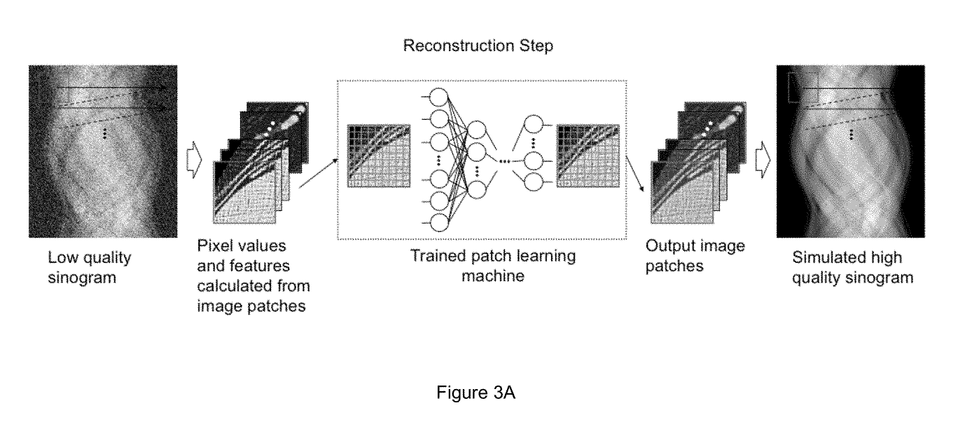

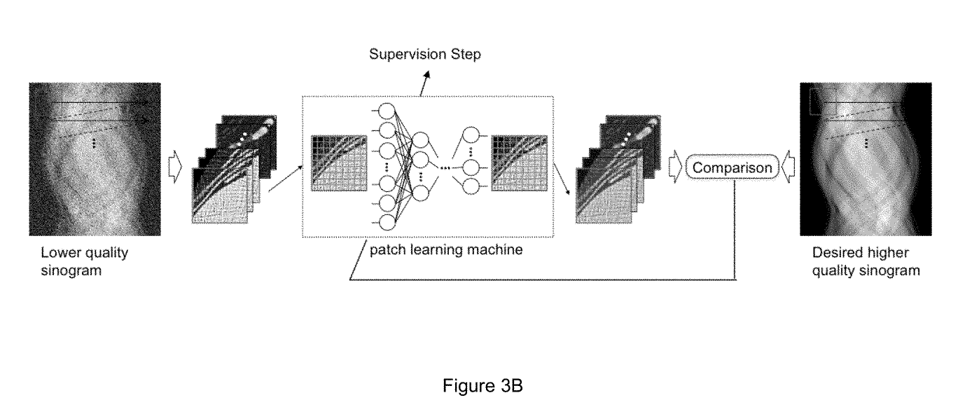

FIG. 3A shows an example of a detailed architecture of the machine-learning model that uses a patch learning machine. The machine-learning model may be a pixel-based machine learning, the formulation of which is described in [37], a regression model such as an artificial neural network regression model, the formulation of which is descried in [38]), (see, for example pages 84-87 in [38]), a support vector regression model, the formulation and theory of which is descried in [39] (see, for example pages 549-558 in [39]), and a nonlinear Gaussian process regression model, the formulation and theory of which is descried in [40]. Other regression models or machine-learning models may be used such as a nearest neighbor algorithm, association rule learning, inductive logic programming, reinforcement learning, representation learning, similarity learning, sparse dictionary learning, manifold learning, dictionary learning, boosting, Bayesian networks, case-based reasoning, Kernel machines, subspace learning, Naive Bayes classifiers, ensemble learning, random forest, decision trees, and statistical relational learning. Among the above models, classifier models such as Naive Bayes classifiers, Bayesian networks, random forest, and decision trees can be used in the machine-learning model, but the performance of the machine-learning model may not he as high as the use of a regression model. In a preferred machine-learning model process, first an image patch is extracted from an input lower-quality projection image that may be acquired at a reduced x-ray radiation dose (lower dose). Pixel values in the local window (image patch, region, or subvolume) are entered into the machine-learning model as input. The output of the machine-learning model in this example preferably is a local window (image patch, region, or subvolume) f(x,y,z), represented by

f(x,y,z)=ML{I(x,y,z)}, (4)

I(x,y,z)={g(x−i,y−j,z−k)|i,j,k∈V_{I}}, (5)

f(x,y,z)={f(x−i,y−j,z−k)|i,j,k∈V_{O}}, (6)

where ML( ) is a machine learning model such as a neural network regression model, I(x,y,z) is the input vector representing the input local window, f(x,y,z) is the output vector representing the output local window, x, y and z are the image coordinates, g(x,y,z) is an input projection volume, V_{I} is an input local window, V_{O} is an output local window, and i, j and k are variables. An output projection volume o(x,y,z) is obtained by processing the output local window f(x,y,z) with an operation OP, represented by

O(x,y,z)=OP{f(x,y,z)}. (7)

The operation OP converts the output vector into a single scalar value O(x,y,z), which can be averaging, maximum voting, minimum voting, or a machine learning model. Collection of the single scalar values forms an output volume O(x,y,z).

Typically, the size of the output local window is smaller than or equal to that of the input local window. The output local window is as small as a single pixel. With the smallest output local window, the output of the machine-learning model in this example is a single pixel O(x,y,z) that corresponds to the center pixel in the input local window, represented by

O(x,y,z)=ML{I(x,y,z)}, (8)

I(x,y,z)={g(x−i,y−j,z−k)|i,j,k∈V_{I}}. (9)

To locate the center of the local window accurately, the size of the local window is preferably an odd number. Thus, the size of the local window may be 3×3, 5×5, 7×7, 9×9, 11×11, 13×13, 15×15 pixels or larger. However, the size of the local window can be an even number, such as 2×2, 4×4, and 5×5 pixels. The local window preferably is a circle but other array shapes can be used, such as square, rectangular or rounded. To obtain an entire output image, each pixel in the output pixel is transformed by using the trained machine-learning model. Converted pixels outputted from the trained machine-learning model are arranged and put into the corresponding pixel positions in the output image, which forms an output high-quality projection image.

FIG. 3B shows supervision of a machine-learning model. First, a number of image patches together with the corresponding desired pixel values are acquired from the input lower-quality (e.g., lower-dose) projection images and desired higher-quality (e.g., higher-dose) projection images, respectively. Input vectors are calculated from the image patches (extracted by using the local window). The input vectors are then entered to the machine-learning model as input. Output pixel values from the machine-learning model are calculated based on the current parameters in the model. Then, the output pixel values are compared with the corresponding desired pixel values in the desired projection images, and the difference “d” between the two is calculated, for example, represented by

$$d = {\sum }_{p}^{ } {\{ {D}^{( p )} - {O}^{( p )} \}}^{2} , \qquad ( 10 )$$

where D is the p-th pixel value in the desired output image/volume, and 0 is the p-th pixel value in the output projection image/volume.

The parameters in the machine-learning model are adjusted so as to minimize or at least reduce the difference. A method to minimize the difference between the output and the desired value under the least square criterion [41] may be used to adjust the machine-learning model. See, for example page 34 in [41]. The difference calculation and the adjustment are repeated. As the adjustment proceeds, the output pixel values and thus the output projection images become closer to the corresponding desired higher-quality (e.g., higher-dose) projection images. When a stopping condition is fulfilled, the adjustment process is stopped. The stopping condition may be set as, for example, (a) an average difference is smaller than a predetermined difference, or (b) the number of adjustments is greater than a predetermined number of adjustments. After training, the machine-learning model would output higher-quality (e.g., high-dose-like) projection images where image degradation factors such as much noise, many artifacts, and much blurriness due to low radiation dose are substantially reduced (or improved).

It is expected that higher-quality (high-dose-like) projection images look like desired (or gold-standard) high-quality (e.g., real high-dose) projection images. Then, the output higher-quality (e.g., high-dose-like x-ray) projection images (e.g., sinograms) are subject to a reconstruction algorithm such as filtered back-projection, inverse radon transform, iterative reconstruction (IR), or algebraic reconstruction technique (ART). The reconstruction algorithm reconstructs tomographic images from the output high-dose-like x-ray projection images (e.g., sinograms). The reconstructed tomographic images are similar to high-dose CT images, or simulated high-dose CT images, where noise or artifacts are removed, or at least substantially reduced. With the higher-quality projection images, the detectability of lesions and clinically important findings such as cancer can be improved.

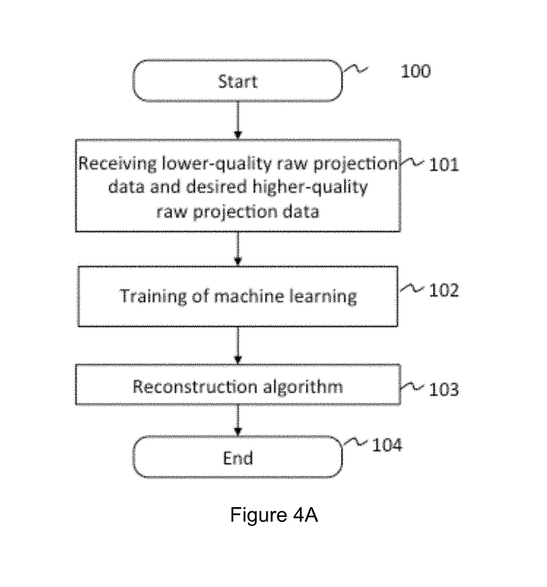

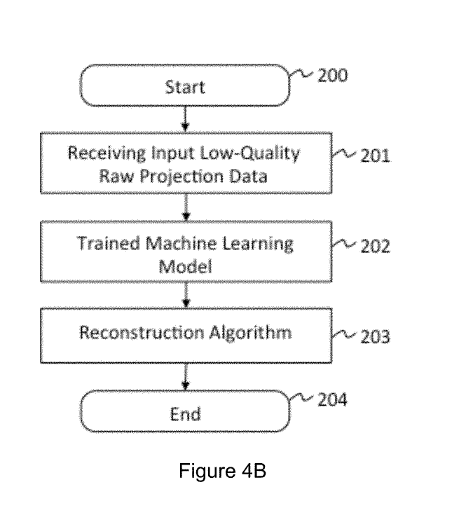

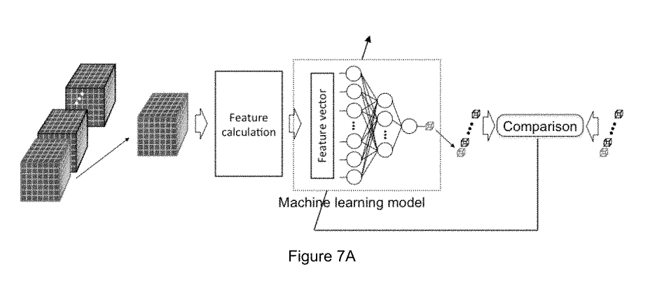

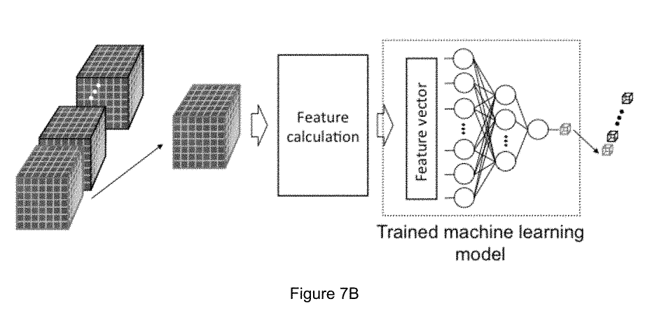

FIG. 4A shows a flow chart for a supervision step of a machine-learning-based transformation. First, in step 101, the machine-learning model receives input lower-dose (raw) projection images with much noise and the corresponding desired higher-dose (raw) projection images with less noise or artifact, which are ideal or desired images to the input lower-dose projection images. In other words, the input projection images are of lower image quality, and the desired projection images are of higher image quality. Regions (image patches or subvolumes) are acquired from the input projection images, and the corresponding regions (image patches or subvolumes) are acquired from the desired projection images. Typically, the size of the desired regions is smaller than or equal to that of the input regions. Typically, the center of the desired region corresponds to the center of the input region. For example, when the input region (image patch) has 3×3 pixels, and the desired region (image patch) is a single pixel, the corresponding location of the desired pixel is located at the second row and the second column in the image patch. Pixel values in the region (image patch) form an N-dimensional input vector where N is the number of pixels in the region (image patch). As shown in FIG. 7A, another very important example is using features extracted from local regions (image patches) (which are not necessarily the same as the input image patches) as input. When a larger patch size is used, features of more global information are extracted. In other words, the extracted features form an N-dimensional input vector or a set of the pixel values and the extracted features form an N-dimensional input vector.

In step 102, the N-dimensional input vector is entered to the machine-learning model as input. The machine-learning model may be a regression model such as an artificial neural network regression model or some other practical regression model. Given the input vector, the machine-learning model with the current set of parameters outputs some output values that form an output image patch. The output image patch and its desired image patch extracted from the desired projection image are compared. The comparison can be done by taking a difference between them, calculating a similarity between them, or taking other comparison measures. The difference may be defined as a mean absolute error, a mean squared error, and a Mahalanobis distance measure. The similarity may be defined as a correlation coefficient, an agreement measure, structural similarity index, or mutual information. In the case of the output image patch being a single pixel, the output pixel value and its desired pixel value obtained from the desired projection image is compared. The machine-learning model may be a pixel-based machine learning, the formulation of which is described in [37], a regression model such as an artificial neural network regression model, the formulation of which is descried in [38]) (see, for example pages 84-87 in [38]), a support vector regression model, the formulation and theory of which is descried in [39] (see, for example pages 549-558 in [39]), and a nonlinear Gaussian process regression model, the formulation and theory of which is descried in [40]. Other regression models or machine-learning models may be used such as a nearest neighbor algorithm, association rule learning, inductive logic programming, reinforcement learning, representation learning, similarity learning, sparse dictionary learning, manifold learning, dictionary learning, boosting, Bayesian networks, case-based reasoning, Kernel machines, subspace learning, Naive Bayes classifiers, ensemble learning, random forest, decision trees, and statistical relational learning. Parameters in the machine-learning model are adjusted so as to maximize similarity or minimize or at least reduce the difference. The adjustment may be made by using an optimization algorithm such as the gradient descent method (such as the steepest descent method and the steepest accent method), conjugate gradient method, or Newton's method. When an artificial neural network regression model is used as the regression model in the machine-learning model, the error-back propagation algorithm [42] can be used to adjust the parameters in the model, i.e., weights between layers in the artificial neural network regression model. The error-back propagation algorithm is an example of the method for adjusting the parameters in the artificial neural network regression model. The formulation and derivation of the error-back propagation algorithm are described in [42] in detail. See, for example pages 161-175 in [42].

In step 103, the output projection images are subject to a reconstruction algorithm such as back-projection, filtered back-projection (FBP), inverse Radon transform, Fourier-domain reconstruction, iterative reconstruction (IR), maximum likelihood expectation maximization reconstruction, statistical reconstruction techniques, polyenergetic nonlinear iterative reconstruction, likelihood-based iterative expectation-maximization algorithms, algebraic reconstruction technique (ART), multiplicative algebraic reconstruction technique (MART), simultaneous algebraic reconstruction technique (SART), simultaneous multiplicative algebraic reconstruction technique (SMART), pencil-beam reconstruction, fan-beam reconstruction, cone-beam reconstruction, sparse sampling reconstruction, and compress sensing reconstruction. The tomographic reconstruction algorithm reconstructs (N+1) dimensional data from N dimensional projection data. It reconstructs simulated high-dose 3D volume with less noise or artifacts from the simulated high-dose 2D projection images, or it reconstructs a simulated high-dose 2D image with less noise or artifacts from a simulated high-dose sinogram.

FIG. 4B shows a flow chart for a reconstruction step of the machine-learning model. This step is performed after the supervision step in FIG. 4A. First, in step 201, the trained machine-learning model receives input low-dose, low-quality (raw) projection images with much noise. Image patches are extracted from input low-dose projection images that are different from the lower-dose projection images used in the supervision step. Pixel values in the image patch form an N-dimensional input vector where N is the number of pixels in the image patch. As shown in FIG. 7A, another very important example is using features extracted from local regions (image patches) (which are not necessarily the same as the input image patches) as input. When a larger patch size is used, features of more global information are extracted. In other words, the extracted features form an N-dimensional input vector or a set of the pixel values and the extracted features form an N-dimensional input vector.

In step 202, the N dimensional input vectors comprising pixel values in the image patches and the features extracted image patches (size of which may be different from that of the former image patches) are entered to the trained machine-learning model as input, and the trained machine-learning model outputs output pixels or output patches. The output patches are converted into output pixels by using a conversion process such as averaging, maximum voting, minimum voting, or a machine-learning model. The output pixels arc arranged and put at the corresponding locations in the output image to form a high-dose-like projection image or a simulated high-dose projection image where noise and artifacts due to low radiation dose are reduced substantially. Thus, the designed machine-learning model provides high-quality simulated high-dose projection images.

In step 203, the high-quality output projection images are subject to a reconstruction algorithm such as back-projection, filtered back-projection (FBP), inverse Radon transform, Fourier-domain reconstruction, iterative reconstruction (IR), maximum likelihood expectation maximization reconstruction, statistical reconstruction techniques, polyenergetic nonlinear iterative reconstruction, likelihood-based iterative expectation-maximization algorithms, algebraic reconstruction technique (ART), multiplicative algebraic reconstruction technique (MART), simultaneous algebraic reconstruction technique (SART), simultaneous multiplicative algebraic reconstruction technique (SMART), pencil-beam reconstruction, fan-beam reconstruction, cone-beam reconstruction, sparse sampling reconstruction, and compress sensing reconstruction. The tomographic reconstruction algorithm reconstructs simulated high-dose 3D volume with less noise or artifacts from the simulated high-dose 2D projection images, or it reconstructs a simulated high-dose 2D image with less noise or artifacts from a simulated high-dose sinogram.

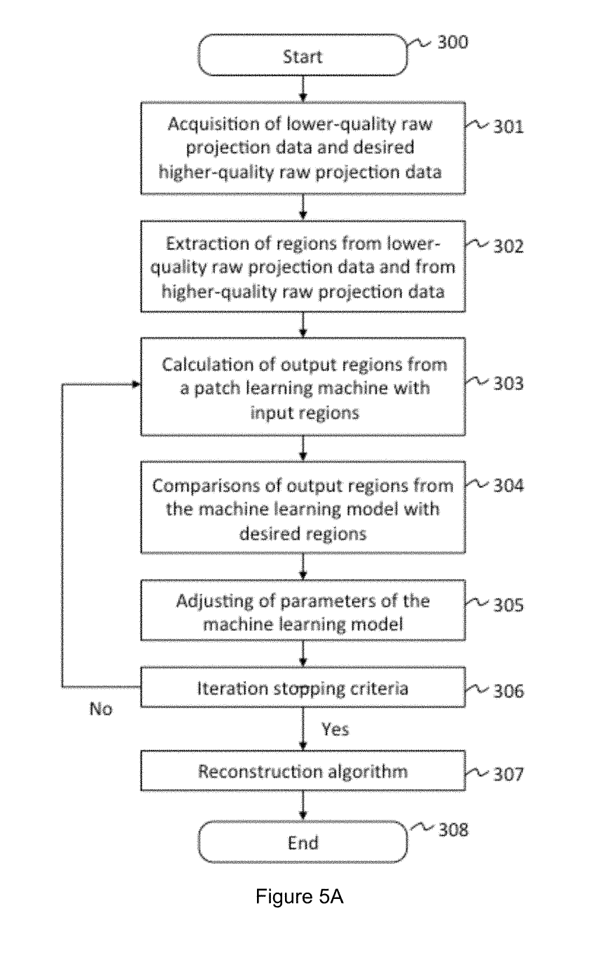

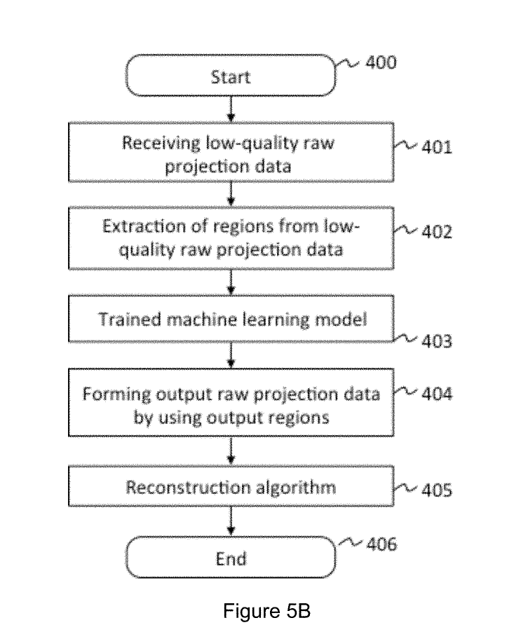

FIG. 5A shows a flow chart for an example of a supervision step of the machine-learning-based transformation. In step 301, the machine-learning model receives input lower-dose, lower-quality (raw) projection images with much noise and artifact and the corresponding desired higher-dose, higher-quality (raw) projection images with less noise or artifact, which are ideal or desired images to the input lower-dose projection images. In step 302, image patches (regions or subvolumes) are acquired from the input lower-dose, lower-quality projection images, and the corresponding image patches (regions or subvolumes) are acquired from the desired higher-dose, higher-quality projection images. In step 303, pixel values in the image patch form an N-dimensional input vector where N is the number of pixels in the image patch. In another example, features are extracted from local image patches (which are not necessarily the same input image patches), and a set of the extracted features or a set of the pixel values and the extracted features form an N-dimensional input vector.

In step 303, the N-dimensional input vector is entered to the machine-learning model as input. In step 304, the output image patches and their desired image patches extracted from the desired projection images are compared. The comparison can be done by taking a difference between them, calculating a similarity between them, or taking other comparison measures. In step 305, parameters in the machine-learning model are adjusted so as to maximize similarity or minimize or at least reduce the difference. In step 306, when a predetermined stopping condition is met, the training is stopped; otherwise it goes back to step 303. The stopping condition may be set as, for example, (a) the average difference (or similarity) is smaller (or higher) than a predetermined difference (or similarity), or (b) the number of adjustments is greater than a predetermined number of adjustments.

In step 307, the output projection images are subject to a reconstruction algorithm such as back-projection, filtered back-projection (FBP), inverse Radon transform, Fourier-domain reconstruction, iterative reconstruction (IR), maximum likelihood expectation maximization reconstruction, statistical reconstruction techniques, polyenergetic nonlinear iterative reconstruction, likelihood-based iterative expectation-maximization algorithms, algebraic reconstruction technique (ART), multiplicative algebraic reconstruction technique (MART), simultaneous algebraic reconstruction technique (SART), simultaneous multiplicative algebraic reconstruction technique (SMART), pencil-beam reconstruction, fan-beam reconstruction, cone-beam reconstruction, sparse sampling reconstruction, and compress sensing reconstruction. The tomographic reconstruction algorithm reconstructs simulated high-dose 3D volume with less noise or artifact from the simulated high-dose 2D projection images, or it reconstructs a simulated high-dose 2D image with less noise or artifacts from the simulated high-dose sinogram.

FIG. 5B shows a flow chart of an example of a reconstruction step of a machine-learning model. In step 401, the trained machine-learning model receives input low-dose, low-quality (raw) projection images with much noise and artifact. In step 402, image patches are extracted from the input low-dose, low-quality projection images. Pixel values in the image patch form an N-dimensional input vector. In another important example, features are extracted from local image patches (which are not necessarily the same input image patches), and a set of the extracted features or a set of the pixel values and the extracted features form an N-dimensional input vector.

In step 403, the N dimensional input vectors comprising pixel values in the image patches and the features extracted image patches (size of which may be different from that of the former image patches) are entered to the trained machine-learning model as input, and the trained machine-learning model outputs output pixels or output patches. The output patches are converted into output pixels by using a conversion process such as averaging, maximum voting, minimum voting, or a machine-learning model. The output pixels are arranged and put at the corresponding locations in the output image to form a high-dose-like projection image (or a simulated high-dose projection image) where noise and artifacts due to low radiation dose are reduced substantially. Thus, the designed machine-learning model provides high-quality simulated high-dose projection images. In step 403, the high-quality output projection images are subject to a reconstruction algorithm such as back-projection, filtered back-projection (FBP), inverse Radon transform, Fourier-domain reconstruction, iterative reconstruction (IR), maximum likelihood expectation maximization reconstruction, statistical reconstruction techniques, polyenergetic nonlinear iterative reconstruction, likelihood-based iterative expectation-maximization algorithms, algebraic reconstruction technique (ART), multiplicative algebraic reconstruction technique (MART), simultaneous algebraic reconstruction technique (SART), simultaneous multiplicative algebraic reconstruction technique (SMART), pencil-beam reconstruction, fan-beam reconstruction, cone-beam reconstruction, sparse sampling reconstruction, and compress sensing reconstruction. The tomographic reconstruction algorithm reconstructs simulated high-dose 3D volume with less noise or artifacts from the simulated high-dose 2D projection images, or it reconstructs a simulated high-dose 2D image with less noise or artifacts from the simulated high-dose sinogram.

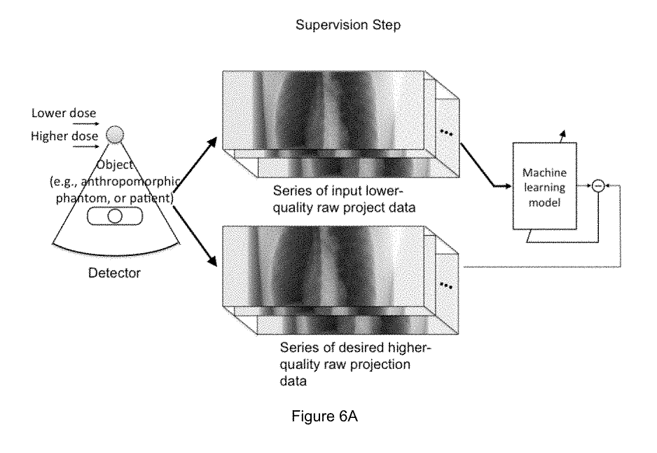

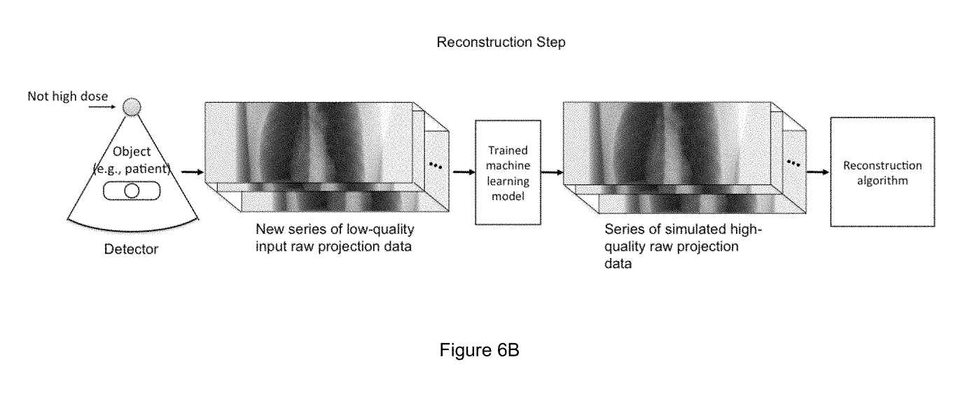

FIG. 6A shows a schematic diagram of the machine-learning-based transformation in a supervision step when a series of 2D raw projection images are acquired from a system. In the same way as in FIG. 2A, the machine-learning model is supervised with input lower-quality (e.g., lower-dose) projection images with high image degradation factors (e.g., much noise, many artifacts, and much blurriness, and low-contrast and low-sharpness) and the corresponding desired higher-dose projection images with improved image degradation factors (e.g., less noise, less artifact, or less blurriness and/or high-contrast and high sharpness). Through the supervision process, the machine-learning model learns to convert lower-quality (e.g., lower-dose) projection images with much noise, more artifacts, and much blurriness, low-contrast and low-sharpens to higher-quality-like (e.g., higher-dose-like) projection images with improved image-degradation factors (e.g. less noise, less artifact, less blurriness, high-contrast, and/or high-sharpness).

FIG. 6B shows a schematic diagram of a machine-learning-based transformation in a reconstruction step when a series of 2D raw projection images are acquired from a system. In the same way as in FIG. 2B, when a new lower-quality (e.g., reduced radiation dose or low dose) projection image is entered to the trained machine-learning model, it would output a projection image similar to its desired projection image, in other words, it would output high-quality (e.g., high-dose-like) projection images (or simulated high-dose projection images) where image degradation factors such as noise, artifact, and blurriness due to low radiation dose are substantially reduced (or improved).

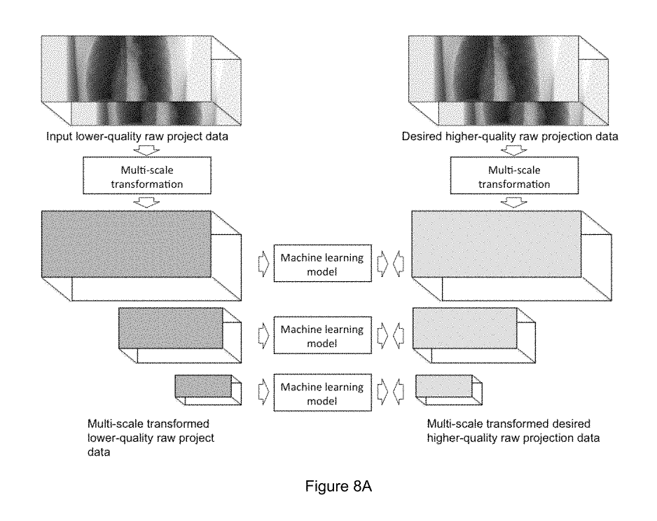

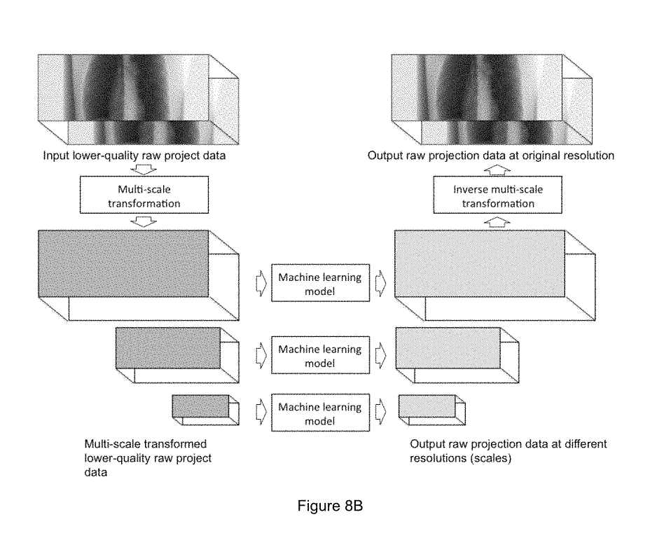

FIG. 8A shows a schematic diagram of a multiple-machine-learning-based transformation in a supervision step with a multi-resolution approach. With a multi-resolution approach, the machine learning models provide high-quality images for wide range of resolutions (scales or sized) objects, namely, from low-resolution objects (or bigger objects) to high-resolution objects (or smaller objects). Lower-dose, lower-quality input raw projection images and the corresponding higher-dose, higher-quality desired raw projection images are transformed by using multi-scale or multi-resolution transformation such as pyramidal multi-resolution transformation, Laplacian pyramids, Gaussian pyramids, and wavelet-based multi-scale decomposition. With the multi-scale approach, the original resolution (scale) input and desired projection images are divided into N different multi-resolution (multi-scale) projection images. N multiple machine learning models are trained with a set of corresponding resolution (scale) input and desired projection images. FIG. 8B shows a schematic diagram of a multiple-machine-learning-based transformation in a reconstruction step with a multi-resolution approach. Lower-dose, lower-quality input raw projection images are transformed by using multi-scale (or multi-resolution) transformation. With the multi-scale approach, the original resolution (scale) input projection images are divided into N different multi-resolution (multi-scale) projection images. Those images are entered to the trained N multiple machine learning models. The output multi-resolution (scale) projection images from the trained N multiple machine learning models are combined by using the inverse multi-resolution (scale) transform to provide the original resolution (scale) simulated high-dose output projection images.

In another implementation example of training the machine-learning model, simulated lower-dose projection images may be used instead of using real lower-dose projection images. This implementation starts with higher-dose projection images with less noise. Simulated noise is added to the higher-dose projection images. Noise in projection images has two different types of noise components: quantum noise and electronic noise. Quantum noise in x-ray images can be modeled as signal-dependent noise, while electronic noise in x-ray images can be modeled as signal-independent noise. To obtain simulated lower-dose projection images, simulated quantum noise and simulated electronic noise are added to the higher-dose projection images.

The input lower-dose projection images and the desired higher-dose projection images preferably correspond to each other, namely, the location and orientation of objects are the same or very close in both images. This can be accomplished easily when a phantom is used. In some examples, the correspondence may be essentially exact, e.g., the lower-dose and higher-dose projection images taken at the same time or right after one another of the same patient or a phantom. In other examples, the lower-dose and higher-dose projection images may be taken at different magnifications or different times. In such cases, an image registration technique may be needed and used to match the locations of objects in the two projection images. The image registration may be rigid registration or non-rigid registration.

Projection images discussed here may be projection images taken on a medical, industrial, security, and military x-ray computed tomography (CT) system, a CT system with a photon counting detector, a CT system with a flat-panel detector, a CT system with single or multiple raw detector, a limited angle x-ray tomography system such as a tomosynthesis, a positron emission tomography (PET) system, a single photon emission computed tomography (SPECT) system, a magnetic resonance imaging (MRI) system, an ultrasound (US) imaging system, an optical coherent tomography system, or their combination.

### Experiments and Evaluation

To prove that an example of machine-learning model works, I developed a machine-learning model to reduce radiation dose in the reconstruction domain in CT. To train and evaluate the machine-learning-based radiation dose reduction, 6 CT scans of an anthropomorphic chest phantom (Kyoto Kagaku, Kyoto, Japan) at 6 different radiation dose levels (0.08, 0.25, 0.47, 1.0, 1.5, and 3.0 mSv) with a CT scanner. The radiation doses were changed by changing tube current-time product, while the tube voltage was fixed at 120kVp. The tube current-time products and the corresponding tube currents in the acquisitions were as follows: 3.5, 10, 17.5, 40, 60 and 120 mAs; 8.8, 25, 44, 100, 150, and 300 mA, respectively. Other scanning and reconstruction parameters were as follows: slice thickness was 0.5 mm; and reconstructed matrix size was 512×512 pixels. The machine learning model was trained with input raw projection images from the 0.08 mSv ultra-low-dose CT scan and the corresponding desired teaching 3.0 mSv high-dose CT scan of the phantom. Contrast-to-noise ratio (CNR) and improvement in CNR (ICNR) were used to measure the image quality of reconstructed CT images. Regression analysis showed that radiation dose was directly proportional to the square of the CNR approximately. The trained machine learning model was applied to a non-training ultra-low-dose (0.08 mSv) projection image, The trained machine learning model provided simulated high-dose (HD) images, The simulated HD images as well as ultra-low-dose real HD projection images were subject to the filtered back projection (FBP) reconstruction algorithm (Lung kernel). The FBP provided simulated HD reconstructed tomographic images.

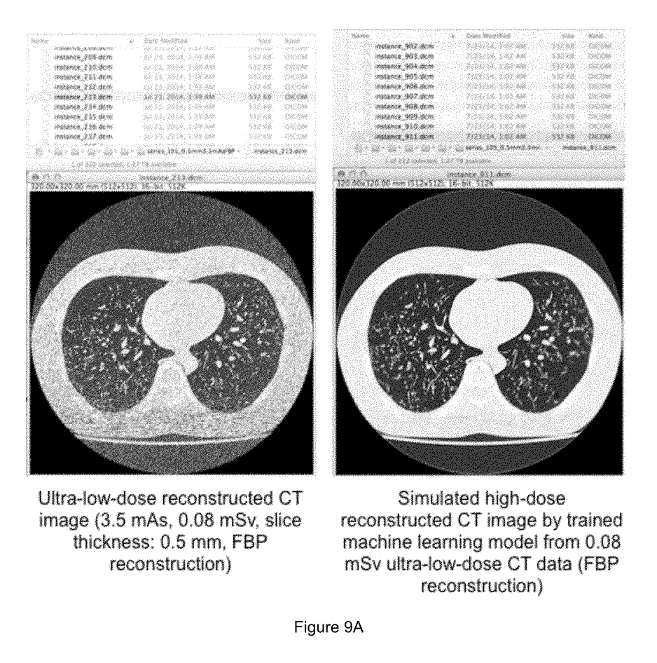

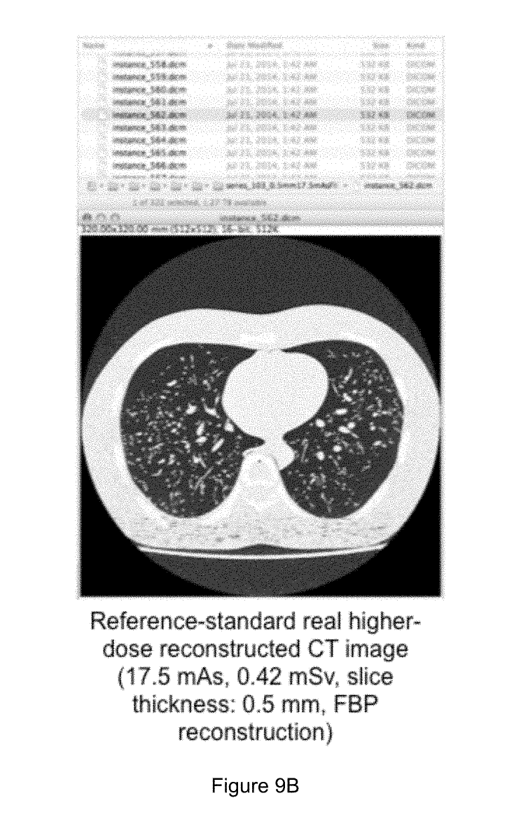

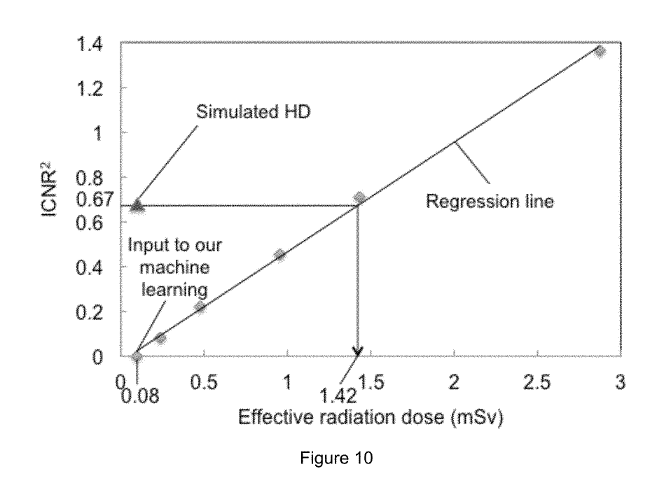

The input ultra-low-dose (0.08 mSv) reconstructed image, simulated HD reconstructed image obtained by the technique in this present invention, and reference-standard real higher-dose (0.42 mSv) reconstructed image are illustrated in FIGS. 9A and 9B. The trained machine-learning-based dose reduction technology reduced noise and streak artifacts in ultra-low-dose CT (0.08 mSv) substantially, while maintaining anatomic structures such as lung vessels, as shown in FIG. 9A. The simulated HD reconstructed CT images are equivalent to the real HD reconstructed CT images, as shown in FIGS. 9A and 9B. The improvement in CNR of the simulated HD reconstructed images from the input ultra-low-dose (0.08 mSv) reconstructed images was 0.67, which is equivalent to 1.42 mSv real HD reconstructed images, as shown in FIG. 10. This result demonstrated 94% (1−0.08/1.42) radiation dose reduction with the developed technology. Thus, the study results with an anthropomorphic chest phantom demonstrated that the machine-learning-based dose reduction technology in the reconstruction domain would be able to reduce radiation dose by 94%.

The processing time for each case was 48 sec. on an ordinary single-core PC (AMD Athlon, 3.0 GHz). Since the algorithm is parallelizable, it can be shortened to 4.1 sec. on a computer with 2 hexa-core processors, and shortened further to 0.5 sec. with a graphics processing unit (GPU).

The machine-learning-based dose reduction technology described in this patent specification may be implemented in a medical imaging system such as an x-ray CT system, a CT system with a photon counting detector, a CT system with a flat-panel detector, a CT system with single or multiple raw detector, a limited angle x-ray tomography system such as a tomosynthesis, a positron emission tomography (PET) system, a single photon emission computed tomography (SPECT) system, a magnetic resonance imaging (MRI) system, an ultrasound (US) imaging system, an optical coherent tomography system, or their combination. The machine-learning-based dose reduction technology may be implemented in a non-medical imaging system such as industrial, security, and military tomographic imaging systems. The machine-learning-based dose reduction technology may be implemented in a computer system or a viewing workstation. The machine-learning-based dose reduction technology may be coded in software or hardware. The machine-learning-based dose reduction technology may be coded with any computer language such as C, C++, Basic, C#, Matlab, python, Fortran, Assembler, Java, and IDL. The machine-learning-based dose reduction technology may be implemented in the Internet space, cloud-computing environment, or remote-computing environment. Converted images from the machine-learning-based dose reduction technology may be handled and stored in the Digital Imaging and Communications in Medicine (DICOM) format, and they may be stored in a picture archiving and communication system (PACS).

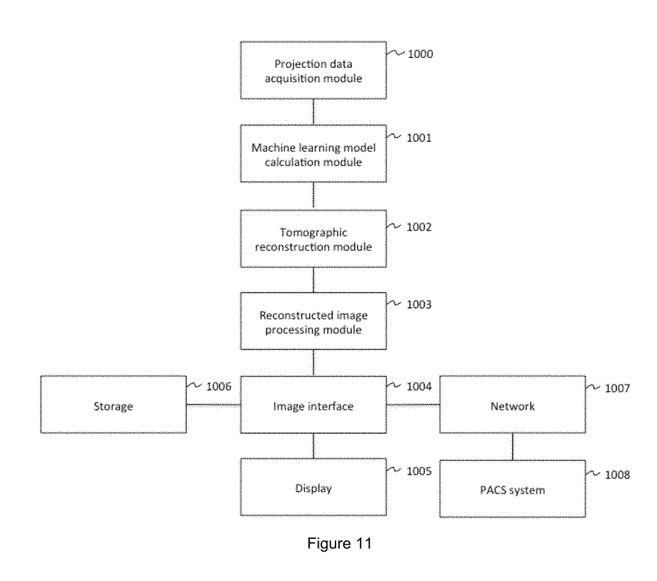

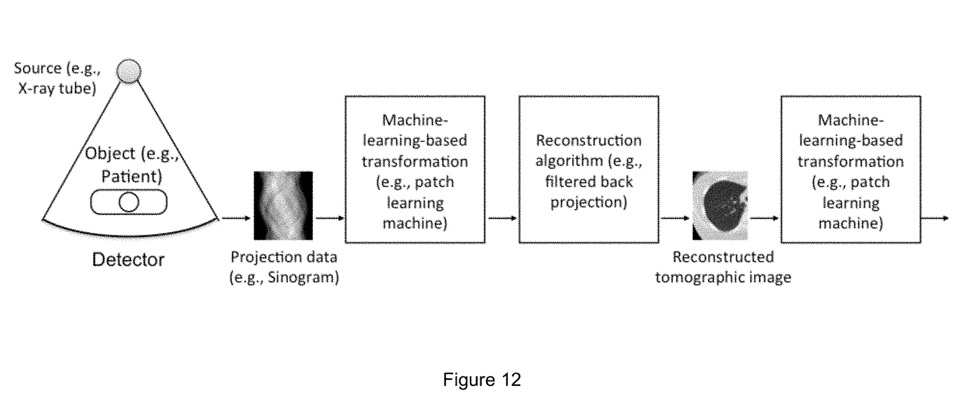

FIG. 11 illustrates an exemplary block diagram of a system that trains the machine learning-based transformation or using a trained machine learning model in the form of a computer. In the reconstruction step, projection data acquisition module 1000 which acquires projection data by rotating a source (such as x-ray source) or an object (such as a patient) provides lower image quality input projection images, such as projection images taken at a lower radiation dose than the standard radiation dose. Projection data acquisition module 1000 can be a medical, industrial, security, and military x-ray computed tomography (CT) system, a limited angle x-ray tomography system such as a tomosynthesis, a positron emission tomography (PET) system, a single photon emission computed tomography (SPECT) system, a magnetic resonance imaging (MRI) system, an ultrasound (US) imaging system, an optical coherent tomography system, or their combination. Machine learning model calculation module 1001 is programmed and configured to apply the processes described above to convert input projection images into output projection images that have higher image quality, and supplies the output projection images to tomographic reconstruction module 1002. The parameters in the machine-learning model may be pre-stored in module 1001. Tomographic reconstruction module 1002 reconstructs higher-quality tomographic images from the output projection images of higher image quality. The higher-quality reconstructed tomographic images are entered into reconstructed image processing module 1003. In reconstructed image processing module 1003, image processing such as further noise reduction, edge enhancement, gray scale conversion, object recognition, or machine-leaning-based image conversion may be performed. In one example, tomographic reconstruction module 1002 directly provides high-quality reconstructed tomographic images to image interface 1004. In another example, reconstructed image processing module 1003 provides high-quality reconstructed tomographic images to image interface 1004. Image interface 1004 provides the tomographic images to storage 1006. Storage 1006 may be a hard drive, RAM, memory, solid-state drive, magnetic tape, or other storage device. Image interface 1004 provides also the tomographic images to display 1005 to display the images. Display 1005 can be a CRT monitor, an LCD monitor, an LED monitor, a console monitor, a conventional workstation commonly used in hospitals to view medical images provided from the DICOM PACS facility or directly from a medical imaging device or from some other source, or other display device. Image interface 1004 provides also the tomographic images to network 1007. Network 1007 can be a LAN, a WAN, the Internet, or other network. Network 1007 connects to a PACS system such as hospital DICOM PACS facility.

In the supervision step, projection data acquisition module 1000 provides lower image quality input projection images, such as projection images taken at a lower radiation dose, and desired teaching higher-quality projection images, such as projection images taken at a higher radiation dose. Machine learning model calculation module 1001 is trained with the above described lower-quality input projection images and desired higher-quality projection images. The desired projection images may be actual projection images taken at a radiation dose that is higher than that used to take the input projection images. Each input projection image is paired with a respective desired projection image. The training in machine learning model calculation module 1001 is done so that output projection images from the machine learning model are closer or similar to the desired higher-quality projection images. For example, the output projection image with the respective desired projection image are compared, and then parameters of the machine learning model are adjusted to reduce the difference between the output projection image and the desired projection image. These steps are repeated until the difference is less than a threshold or some other condition is met, such as exceeding a set number of iterations. The parameters in the machine-learning model may be pre-stored in module 1001, and can be updated or improved from time to time by replacement with a new set of parameters or by training with a new set of input lower-quality projection images and desired higher-quality projection images. Machine learning model calculation module 1001 supplies the parameters in the machine learning model to tomographic reconstruction module 1002, reconstructed image processing module 1003, image interface 1004, or directly to storage 1006 or network 1007.

The image transformation and reconstruction processes described above can be carried out through the use of modules 1001 and 1002 that are programmed with instruction downloaded from a computer program product that comprises computer-readable media such as one or more optical discs, magnetic discs, and flash drives storing, in non-transitory form, the necessary instructions to program modules 1001 and 1002 to carry out the described processes involved in training the machine learning model and/or using the trained machine learning model to convert lower image quality input projection images into higher image quality projection images. The instructions can be in a program written by a programmer of ordinary skill in programming based on the disclosure in this patent specification and the material incorporated by reference, and general knowledge in programming technology.

When implemented in software, the software may be stored in any computer readable memory such as on a magnetic disk, an optical disk, or other storage medium, in a RAM or ROM or flash memory of a computer, processor, hard disk drive, optical disk drive, tape drive, etc. Likewise, the software may be delivered to a user or a system via any known or desired delivery method including, for example, on a computer readable disk or other transportable computer storage mechanism or via communication media. Communication media typically embodies computer readable instructions, data structures, program modules or other data in a modulated data signal such as a carrier wave or other transport mechanism. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Thus, the software may be delivered to a user or a system via a communication channel such as a telephone line, a DSL line, a cable television line, a wireless communication channel, the Internet, etc. (which are viewed as being the same as or interchangeable with providing such software via a transportable storage medium).

While numerous specific details are set forth in the following description in order to provide a thorough understanding, some embodiments can be practiced without some or all of these details. While several embodiments are described, it should be understood that the technology described in this patent specification is not limited to any one embodiment or combination of embodiments described herein, but instead encompasses numerous alternatives, modifications, and equivalents.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

For the purpose of clarity, certain technical material that is known in the related art has not been described in detail in order to avoid unnecessarily obscuring the new subject matter described herein. It should be clear that individual features of one or several of the specific embodiments described herein can be used in combination with features or other described embodiments.

The various blocks, operations, and techniques described above may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

Like reference numbers and designations in the various drawings indicate like elements. There can be alternative ways of implementing both the processes and systems described herein that do not depart from the principles that this patent specification teaches. Accordingly, the present embodiments are to be considered as illustrative and not restrictive, and the body of work described herein is not to be limited to the details given herein, which may be modified within the scope and equivalents of the appended claims.

Thus, although certain apparatus constructed in accordance with the teachings of the invention have been described herein, the scope of coverage of this patent is not limited thereto. On the contrary, this patent covers all embodiments of the teachings of the invention fairly falling within the scope of the appended claims either literally or under the doctrine of equivalents.

What is claimed:

1. A method of processing a projection data, comprising:
obtaining lower quality input projection data from a system;
extracting information from the input projection data;
entering the input information into a machine learning model as input and obtaining output information from the model;
forming output projection data of quality higher than that of the input projection data from the machine learning model;
reconstructing tomographic data from the output projection data.

2. The method of claim 1, wherein the input information is plural regions from the input projection data, features extracted from plural regions in the input projection data, pixels in plural regions in the input projection data, or any combination of them.

3. The method of claim 1, wherein the input projection data are sinograms, two-dimensional images, three-dimensional images, or any combination of them.

4. The method of claim 1, wherein the lower quality input projection data are obtained from a detector of the system.

5. The method of claim 1, wherein the system is a computed tomography system, an x-ray computed tomography system, an optical tomography system, a magnetic resonance imaging system, an ultrasound imaging system, a positron emission tomography system, a single photon emission computed tomography system, or any combination of them.

6. The method of claim 1, wherein the machine learning model is at least one of an artificial neural network, artificial neural network regression, a support vector machine, support vector regression, a shallow convolutional neural network, a deep convolutional neural network, deep learning, a deep brief network, supervised nonlinear regression, supervised nonlinear regression, nonlinear Gaussian process regression, a shift-invariant neural network, a nearest neighbor algorithm, association rule learning, inductive logic programming, reinforcement learning, representation learning, similarity learning, sparse dictionary learning, manifold learning, dictionary learning, boosting, a Bayesian network, case-based reasoning, a Kernel machine, subspace learning, a Naive Bayes classifier, ensemble learning, random forest, decision frees, a bag of visual words, statistical relational learning, or any combination of them.

7. The method of claim 1, wherein the reconstruction of tomographic data is at least one of back-projection, filtered back-projection, inverse Radon transform, Fourier-domain reconstruction, iterative reconstruction, maximum likelihood expectation maximization reconstruction, statistical reconstruction techniques, polyenergetic nonlinear iterative reconstruction, likelihood-based iterative expectation-maximization algorithms, algebraic reconstruction technique, multiplicative algebraic reconstruction technique, simultaneous algebraic reconstruction technique, simultaneous multiplicative algebraic reconstruction technique, pencil-beam reconstruction, fan-beam reconstruction, cone-beam reconstruction, sparse sampling reconstruction, and compress sensing reconstruction, or any combination of them.

8. The method of claim 1, wherein the input projection data are obtained from one or more of a system, computer storage, a viewing workstation, a picture archiving and communication system, cloud computing, website, and the Internet.

9. The method of claim 1, wherein the input projection data are obtained at radiation doses between approximately 1% and 90% of a standard radiation dose.

10. The method of claim 1, wherein the machine learning model is a previously-trained machine learning model that is trained with lower quality projection data and higher quality projection data.

11. The method of claim 11, wherein the lower quality projection data are lower radiation-dose projection data; and the higher quality projection data are higher radiation-dose projection data.

12. A method of processing a projection data, comprising:
obtaining pairs of input projection data and desired projection data from a system;
training a machine learning model with the pairs of the information extracted from the input projection data and the information extracted from desired projection data.

13. The method of claim 12, wherein the pairs of the information are pairs of regions extracted from the input projection data and regions or otherwise pixels extracted from the desired projection data.

14. The method of claim 12, wherein the information extracted from the input projection data are features extracted from plural regions in the input projection data, regions or otherwise pixels in plural regions in the input projection data, or combination of them.

15. The method of claim 12, wherein the training of a machine learning model is done by:
comparing between output information from the machine learning model and corresponding desired information from the desired projection data;
adjusting parameters in the machine learning model based on the comparison.

16. The method of claim 15, wherein the comparison between output information and corresponding desired information is done by calculating a mean absolute error between the output information and the corresponding desired information, or a mean squared error between the output information and the corresponding desired information.

17. The method of claim 15, wherein the adjustment of parameters comprise at least one of an error-back propagation algorithm, a steepest descent method, Newton's algorithm, and an optimization algorithm.

18. The method of claim 12, wherein the input projection data are relatively lower quality projection data; and the desired projection data are relatively higher quality projection data.

19. The method of claim 12, wherein the input projection data are acquired at radiation doses between approximately 0.01% and 90% of the radiation doses at which the desired projection data are acquired.

20. The method of claim 12, wherein the input projection data are sinograms, two-dimensional images, three-dimensional images, or any combination of them; and the desired projection data are desired sinograms, two-dimensional images, three-dimensional images, or any combination of them.

21. The method of claim 12, wherein the system is a computed tomography system, an x-ray computed tomography system, an optical tomography system, a magnetic resonance imaging system, an ultrasound imaging system, a positron emission tomography system, a single photon emission computed tomography system, or any combination of them.

22. The method of claim 12, wherein the machine learning model is at least one of an artificial neural network, artificial neural network regression, a support vector machine, support vector regression, a shallow convolutional neural network, a deep convolutional neural network, deep learning, a deep brief network, supervised nonlinear regression, supervised nonlinear regression, nonlinear Gaussian process regression, a shift-invariant neural network, a nearest neighbor algorithm, association rule learning, inductive logic programming, reinforcement learning, representation learning, similarity learning, sparse dictionary learning, manifold learning, dictionary learning, boosting, a Bayesian network, case-based reasoning, a Kernel machine, subspace learning, a Naive Bayes classifier, ensemble learning, random forest, decision trees, a bag of visual words, statistical relational learning, or any combination of them.

23. The method of claim 12, wherein the input projection data are obtained from one or more of a system, computer storage, a viewing workstation, a picture archiving and communication system, cloud computing, website, and the Internet.

24. A computer program product comprising instructions stored in computer-readable media that, when loaded into and executed by a computer system cause the computer system to carry out the process of:
obtaining lower quality input projection data from a system;
extracting information from the input projection data;
entering the input information into a machine learning model as input and obtaining output information from the model;
forming output projection data of quality higher than that of the input projection data from the machine learning model;
reconstructing tomographic data from the output projection data.
